(19) United States
(12) Patent Application Publication
(10) Pub. No.: US 2007/0276359 A1
(43) Pub. Date: Nov. 29, 2007

(54) Medical laser wand

(76) Inventor: Kim Robin Segal, Plano, TX (US)

Correspondence Address: DOERNER, SAUNDERS, DANIEL & ANDERSON, LLP, 201 Robert S. Kerr Avenue, Suite 700, OKLAHOMA CITY, OK, 73102-4203, US

(21) Appl. No.: 11/441,504

(22) Filed: May 26, 2006

(51) Int. Cl. A61N 5/06; A61B 18/18

(52) U.S. Cl. 606/11; 607/89

(57) ABSTRACT

A diode laser irradiation system for treating biological tissue of a subject without exposing the tissue to damaging thermal effects. The system includes a wand containing one or more diode lasers disposed for irradiating the tissue with coherent optical energy at a power output level of less than 1000 mw in aggregate at a wavelength in a range of from about 2500 nm to about 10,000 nm, and laser setting controls for operating the diode laser to achieve a rate of absorption and conversion to heat in the irradiated tissue in a range between a minimum rate sufficient to elevate the average temperature of the irradiated tissue to a level above the basal body temperature of the subject, and a maximum rate which is less than the rate at which the irradiated tissue is converted into a collagenous substance. The wand may be used manually or automatically in hands-free mode.

### CROSS-REFERENCES TO RELATED APPLICATIONS

This application claims the benefit of U.S. Provisional Patent Application, Ser. No. 60/683,279, filed on May 27, 2005, the contents of which are hereby incorporated by reference in their entirety.

### BACKGROUND OF THE INVENTION

The present invention generally relates to methods and devices for the treatment of living biological tissue, and more particularly to a hands-free laser wand for use in stimulating soft, living tissue by diode laser irradiation.

Various non-surgical means have been employed in the therapeutic treatment of living tissue. Such techniques have included the application of ultrasonic energy, electrical stimulation, high frequency stimulation by diathermy, X-rays, and microwave irradiation. While these techniques have shown some therapeutic benefit, their use has been somewhat limited because they generate excessive thermal energy which can damage tissue. Consequently, the energy levels associated with therapeutic treatments involving diathermy, X-ray, microwave, and electrical stimulation have been limited to such low levels that little or no benefit has been obtained. Moreover, the dosage or exposure to microwaves and X-ray radiation must be carefully controlled to avoid causing health problems related to the radiation they generate. Ultrasonic energy is non-preferentially absorbed and affects all of the tissue surrounding the area to which it is directed.

Optical energy generated by lasers has been used for various medical and surgical purposes because laser light, as a result of its monochromatic and coherent nature, can be selectively absorbed by living tissue. The absorption of the optical energy from laser light depends upon certain characteristics of the wavelength of the light and properties of the irradiated tissue, including reflectivity, absorption coefficient, scattering coefficient, thermal conductivity, and thermal diffusion constant. The reflectivity, absorption coefficient, and scattering coefficient are dependent upon the wavelength of the optical radiation. The absorption coefficient is known to depend upon such factors as interband transition, free electron absorption, grid absorption (photon absorption), and impurity absorption, which are also dependent upon the wavelength of the optical radiation.

In living tissue, water is a predominant component and has, in the infrared portion of the electromagnetic spectrum, an absorption band determined by the vibration of water molecules. In the visible portion of the spectrum, there exists absorption due to the presence of hemoglobin. Further, the scattering coefficient in living tissue is a dominant factor.

Thus, for a given tissue type, the laser light may propagate through the tissue substantially unattenuated, or may be almost entirely absorbed. The extent to which the tissue is heated and ultimately destroyed depends on the extent to which it absorbs the optical energy. It is generally preferred that the laser light be essentially transmissive through tissues which are not to be affected, and absorbed by tissues which are to be affected. For example, when applying laser radiation to a region of tissue permeated with water or blood, it is desired that the optical energy not be absorbed by the water or blood, thereby permitting the laser energy to be directed specifically to the tissue to be treated. Another advantage of laser treatment is that the optical energy can be delivered to the treatment tissues in a precise, well defined location and at predetermined, limited energy levels.

Ruby and argon lasers are known to emit optical energy in the visible portion of the electromagnetic spectrum, and have been used successfully in the field of ophthalmology to reattach retinas to the underlying choroidea and to treat glaucoma by perforating anterior portions of the eye to relieve interoccular pressure. The ruby laser energy has a wavelength of 694 nanometers (nm) and is in the red portion of the visible spectrum. The argon laser emits energy at 488 nm and 515 nm and thus appears in the blue-green portion of the visible spectrum. The ruby and argon laser beams are minimally absorbed by water, but are intensely absorbed by blood chromogen hemoglobin. Thus, the ruby and argon laser energy is poorly absorbed by non-pigmented tissue such as the cornea, lens and vitreous humor of the eye, but is absorbed very well by the pigmented retina where it can then exert a thermal effect.

Another type of laser which has been adapted for surgical use is the carbon dioxide (CO₂) gas laser which emits an optical beam which is absorbed very well by water. The wavelength of the CO₂ laser is 10,600 nm and therefore lies in the invisible, far infrared region of the electromagnetic spectrum, and is absorbed independently of tissue color by all soft tissues having a high water content. Thus, the CO₂ laser makes an excellent surgical scalpel and vaporizer. Since it is completely absorbed, its depth of penetration is shallow and can be precisely controlled with respect to the surface of the tissue being treated. The CO₂ laser is thus used in various surgical procedures in which it is necessary to vaporize or coagulate neutral tissue with minimal thermal damage to nearby tissues.

Another laser in widespread use is the neodymium doped yttrium-aluminum-garnet (Nd:YAG) laser. The Nd:YAG laser has a predominant mode of operation at a wavelength of 1064 nm in the near infrared region of the electromagnetic spectrum. The Nd:YAG optical emission is absorbed to a greater extent by blood than by water and is used for coagulating large, bleeding vessels. The Nd:YAG laser has been transmitted through endoscopes for treatment of a variety of gastrointestinal bleeding lesions, such as esophageal varices, peptic ulcers, and arteriovenous anomalies.

The foregoing applications of laser energy are in use as surgical scalpels and in situations where high energy thermal effects are desired, such as tissue vaporization, tissue cauterization, and coagulation. Although the foregoing laser systems perform well, they commonly generate large quantities of heat and require a number of lenses and mirrors to properly direct the laser light and, accordingly, are relatively large, unwieldy, and expensive. These problems are somewhat alleviated in some systems by locating a source of laser light distal from a region of tissue to be treated and providing fiber optic cable for carrying light generated from the source to the tissue region, thereby obviating the need for a laser light source proximal to the tissue region. Such systems, however, are still relatively large and unwieldy and, furthermore, are much more expensive to manufacture than a system which does not utilize fiber optic cable. Moreover, the foregoing systems generate thermal effects which can damage living tissue, rather then provide therapeutic treatment to the tissue.

Applicant's prior art patents, U.S. Pat. No. 5,755,752 and U.S. Pat. No. 6,033,431, the disclosures of which are incorporated herein by reference, disclose a system and a method that retain all of the advantages of the foregoing systems while reducing the size and cost of the system. This system treats biological tissue without exposing the tissue to damaging thermal effects and provides a wand which houses an Indium Gallium Arsenide (In:GaAs) diode laser configured for generating coherent optical energy radiation having a wavelength in the range of the near infrared region of the electromagnetic spectrum (1064 nm-2500 nm) at a power output in the range about 100 mw-1000 mw. The coherent optical energy radiation is focused on the treatment area to achieve a rate of absorption and conversion to heat in the irradiated tissue in the range between a minimum rate sufficient to elevate the average temperature of the irradiated tissue to a level above the basal body temperature of the living subject, and a maximum rate which is less than the rate at which the irradiated tissue is converted into a collagenous substance. This provides therapeutic medical treatment.

It has been found that such a long wavelength laser diode, operating in the region beyond near infrared region and less than far infrared, i.e. 2500 nm to about 10,000 nm, can be used at much lower power (<100 mw) than heretofore and still provide the same therapeutic, nondestructive effect on human tissue that higher power laser devices provide at shorter wavelengths.

Therefore, what is needed is a system and method of therapeutic tissue stimulation that operates in the infrared region of from about 2500 nm to about 10,000 nm at low power, in order to economically stimulate soft, living tissue with laser energy without damaging the tissue from the thermal effects of that laser energy.

### SUMMARY OF THE INVENTION

It is therefore an object of this invention to provide a therapeutic medical laser operating in the region between near infrared and far infrared, which requires less power to operate than heretofore.

In one aspect, this invention features a diode having a wavelength beyond the near infrared region, operating at low power that is used for medical therapeutic applications.

In another aspect, this invention features one or more of such laser diodes which operate at wavelengths greater than 1064 nm and at a power of less than 100 mw.

These and other features, aspects, and advantages of the present invention will become better understood with reference to the following drawings, description, and claims.

### BRIEF DESCRIPTION OF THE DRAWINGS

FIG. 1 shows a schematic diagram of a multi-diode laser irradiation system, according to an embodiment of the present invention;

FIG. 2 shows a cross-sectional view of a wand used in the system of FIG. 1, according to an embodiment of the present invention;

FIG. 3A shows an enlarged, cross sectional view of a diode array used in the wand of FIG. 2, according to an embodiment of the present invention;

FIG. 3B shows an enlarged, end view of the wand of FIG. 3A, according to an embodiment of the present invention;

FIG. 4 shows a perspective view of a hands-free device for directing the wand in a prescribed pattern over a portion of a patient's skin, according to an embodiment of the present invention; and

FIG. 5 shows a cross-sectional view of a proximity sensor associated with the end of a wand, according to an embodiment of the present invention.

### DETAILED DESCRIPTION OF THE INVENTION

The following detailed description is of the best currently contemplated modes of carrying out the invention. The description is not to be taken in a limiting sense, but is made merely for the purpose of illustrating the general principles of the invention, since the scope of the invention is best defined by the appended claims.

Broadly, the current invention includes systems, devices, and methods for the treatment of living biological tissue, either manually through use of a laser wand for stimulating soft, living tissue by diode laser irradiation, or automatically through use of a hands-free device that directs and controls the laser wand under programmatic control. The laser wand allows laser diodes located in an end of the wand to be moved over a given area of human skin surface. The automatic capability allows the laser wand to be moved in a regular manner, while providing a consistent energy output to the area being traversed, thus providing a more consistent result than when the laser wand is manually moved over a given area of the skin. The results are repeatable from session to session, so that healing progress may be more accurately measured than heretofore.

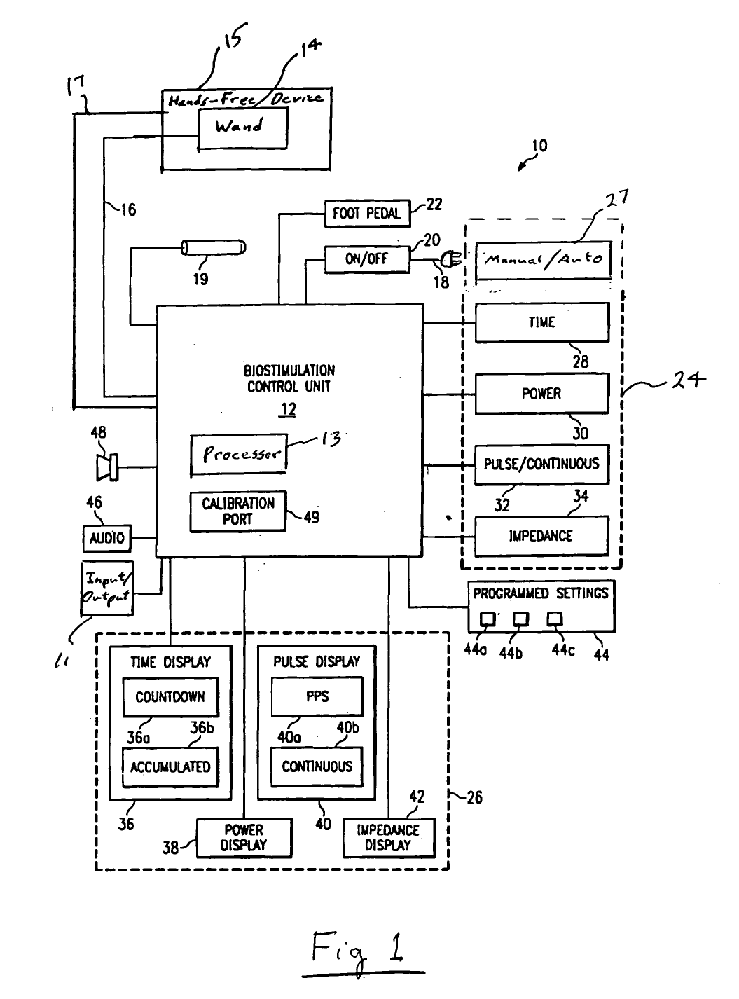

Referring to a block diagram of an embodiment of the invention given in FIG. 1, a diode laser irradiation system 10 is provided, which includes a medical therapeutic biostimulation control unit 12 for selectively controlling the operation of wand 14 and of a hands-free device 15 for manipulating the wand 14. The biostimulation control unit 12 may also contain a processor 13, which may be in the form of a programmable computer, microprocessor, or other computational device that can be dynamically programmed. An input/output device 11 may also be connected to the biostimulation control unit 12. Depending upon different applications of the diode laser irradiation system 10, the input/output device 11 may be used to save parameters manually set up on the laser setting controls 24, i.e. a “protocol”; to retrieve a protocol that has been developed by some external software application; or provide limiting instructions or values when the diode laser irradiation system 10 is being leased for use by third parties.

The biostimulation control unit 12 may receive power through a power supply line 18 that is connected to a conventional 120-volt power outlet. A grounding device 19 may be connected to control unit 12 for holding by a patient receiving the tissue irradiation therapy, in order to provide an electrical ground for safety. An on/off switch 20 may be connected in series with line 18 for controlling the flow of power. A foot pedal 22 may optionally be connected to control unit 12 and depressed to activate the generation and emission of laser light from wand 14.

The wand 14 may be electrically connected to biostimulation control unit 12 via a coaxial cable 16 and manually controlled, and the hands-free device 15 may be electrically connected and controlled via a coaxial cable 17. As described below, the wand 14 may house one or more laser diodes, each emitting low level reactive laser light for use in tissue irradiation therapy. The wand 14 may optionally be removed from the hands-free device 15 so that the operator of the diode laser irradiation system 10 may manually manipulate the wand 14 over a portion of the tissue of a patient receiving irradiation therapy. The hands-free device 15 will be described presently.

Biostimulation control unit 12 may include laser setting controls 24 and corresponding laser setting displays 26. Setting controls 24 may be used to manually select operational parameters of the control unit 12 to affect the rate of absorption and conversion to heat of tissue irradiated by wand 14, according to desired treatment protocols. Generally, these treatment protocols provide for a rate of absorption and conversion to heat in the irradiated tissue in a range between a minimum rate, which is sufficient to elevate the average temperature of the irradiated tissue to a level above the basal body temperature of the subject, and a maximum rate, which is less than the rate at which the irradiated tissue is converted to a collagenous substance. Treatment protocols may vary time, power, and pulse/continuous mode parameters in order to achieve the desired therapeutic effects.

Setting controls 24 may include a manual/automatic mode control 27, treatment time control 28, a power control 30, and a pulse/continuous mode control 32. Adjustments in the treatment time, power, and pulse/continuous mode operation of wand 14 that use the controls 28, 30, and 32 may enable improved therapeutic effects based upon the aforementioned treatment protocols involving one or more of these parameters. An impedance control 34 may adjust the impedance measurement of the tissue to a baseline value, according to skin resistance, in order to monitor improvements in tissue condition. The setting controls 24 of the biostimulation control unit 12 may include any combination of one or more of the controls 27, 28, 30, 32, or 34.

Setting displays 26 may include a time display 36, a power display 38, a pulse display 40, and an impedance display 42, which may be LED displays, meters, or other display devices well known to the art, may enable setting controls 24 to be operated to increment or decrement the settings that are then indicated on the displays. A programmed settings control 44 may be used to save setting selections and then automatically recall them for convenience, using one or more buttons 44a, 44b, or 44c, for example.

The manual/automatic mode control 27 may be used to select the overall mode of operation of the biostimulation control unit 12. When manual mode is selected, then all the remaining controls may be enabled and recognized by the bistimulation control unit 12 for manually setting various parameters necessary to operate the biostimulation control unit 12 according to a given treatment protocol, but the hands-free device 15 may be disabled. When the automatic mode is selected, then all controls may be disabled with the exception of the programmed settings control 44 for recalling a previous setting. The automatic mode may be used when the hands-free device 15 is connected to the biostimulation control unit 12 and enabled.

Time control 28 may adjust the time that laser light is emitted from the wand 14 from 0.01 to 9.99 minutes in 0.01 minute intervals, as indicated on the time display 36. The time display 36 may include a countdown display 36a and an accumulated display 36b. Once the time control 28 is set, the countdown display 36a may indicate the setting so that, as the wand 14 is operated, the time is decremented to zero. The accumulated time display 36b may increment from zero (or any other reset value) as the wand 14 is operated, so that the total treatment time is displayed. The time display 36 may take into account the pulsed or continuous mode operation of the system 10.

Power control 30 may adjust the power dissipation level of the laser light from the wand 14. The pulse/continuous mode control 32 may set the system 10 to generate laser light energy from the wand 14 either continuously or as a series of pulses.

An audio volume control 46 may be provided for generating an audible warning tone from a speaker 48 when laser light is being generated. Thus, for example, the tone may be pulsed when the system is operating in the pulse mode of operation.

Impedance control 34 may be provided as a sensitivity setting that is calibrated and set, according to the tissue skin resistance, to a baseline value that is then indicated on the impedance display 42. As therapy progresses, the impedance readout on display 42 may change (i.e., decrease), thereby indicating progress of treatment.

A calibration port 49 may be provided to verify laser performance by placing the wand 14 in front of the port and operating the system 10 to determine whether the system 10 is operating within calibration specifications, and automatically adjusting the system parameters.

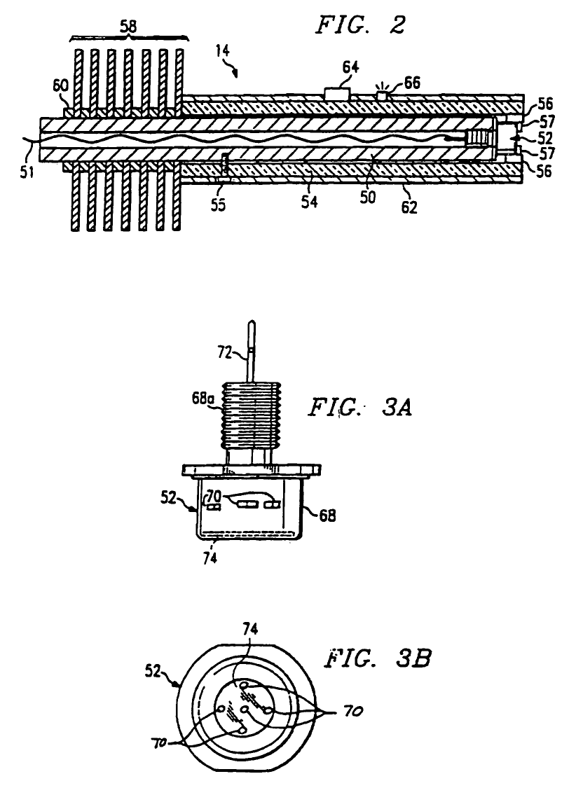

Referring to FIG. 2, wand 14, sized for easy manual manipulation and for articulation by the hands-free device 15, may include a heat-conductive, metal bar 50. Bar 50 may be hollow along its central axis and may be threaded on its interior at a first end for receiving a laser resonator 52, described further below with reference to FIGS. 3A and 3B. Wiring 51 may extend from the laser resonator 52 through hollow axis of bar 50 for connection to the coaxial cable 16 (FIG. 1). In one embodiment, bar 50 may be copper or steel and thus may conduct electricity for providing a ground connection for resonator 52 to cable 16.

A sleeve 54 is placed over bar 50 for electrical and thermal insulation. The sleeve may be composed, for example, of a modified type of polypropylene oxide (PPO) styrene, such as that sold under the trademark Noryl, produced by the General Electric Company, New York, but other similar non-conductive substances may be used without departing from the scope of the invention. A screw 55 extending through sleeve 54 may anchor the sleeve to the bar 50. As shown, the laser resonator 52 may be recessed slightly within sleeve 54. An impedance O-ring 56, formed of a conductive metal, may be press-fitted into the end of sleeve 54 so that when wand 14 makes contact with tissue, ring 56 touches the tissue. Ring 56 may be electrically connected through wand 14 to unit 12. Impedance O-ring 56 may optionally measure impedance by measuring angular DC resistance with an insulator ohmmeter, for example, of the tissue being irradiated by wand 14, which may then be displayed as impedance on the display 42. Impedance O-ring 56 may also optionally be used as a proximity sensor when the wand 14 is being directed by the hands-free device 15 as will be described presently.

Measurements of impedance are useful in therapy to determine whether healing has occurred. For example, a baseline measurement of impedance may provide an objective value of comparison, wherein as the tissue heals, a lower impedance approaching the baseline is observed. The impedance value read can also be used to determine the amount of wattage and time of treatment appropriate for the patient.

A feedback sensor 57 may be located in the end of sleeve 54 for measuring the output of laser resonator 52. While not shown, the feedback sensor 57 may be connected electronically to control unit 12 and to a feedback circuit within control unit 12. A small percentage of the diode laser light from the laser resonator 52 may thus be detected by the feedback sensor 57 and channeled into the feedback circuit of control unit 12 to measure and control performance of resonator 52. Out-of-specification temperature, power, pulse frequency or duration may thus be corrected or the system 10 may be automatically turned off.

Multiple metallic fins 58 may be placed over the end of bar 50 and may be separated and held in place by spacers 60 press-fitted over bar 50. The fins 58 may act as a heat sink to absorb heat from the laser through bar 50 and dissipate the heat into ambient air. Spacers 60 placed between each fin 58 may enable air to flow between the fins, thereby providing for increased heat transfer from wand 14.

A casing 62 may be fitted over sleeve 54 to serve as a hand grip and structure to support a switch 64 and light 66. The switch 64 may actuate wand 14 by the operator when the wand 14 is being manually manipulated by the operator. Switch 64 may be wired in a suitable manner to control unit 12 and used either alone or in conjunction with foot pedal 22 when the wand 14 is being manually manipulated. Light 66 may be illuminated to provide a power-on indication when wand 14 is in operation.

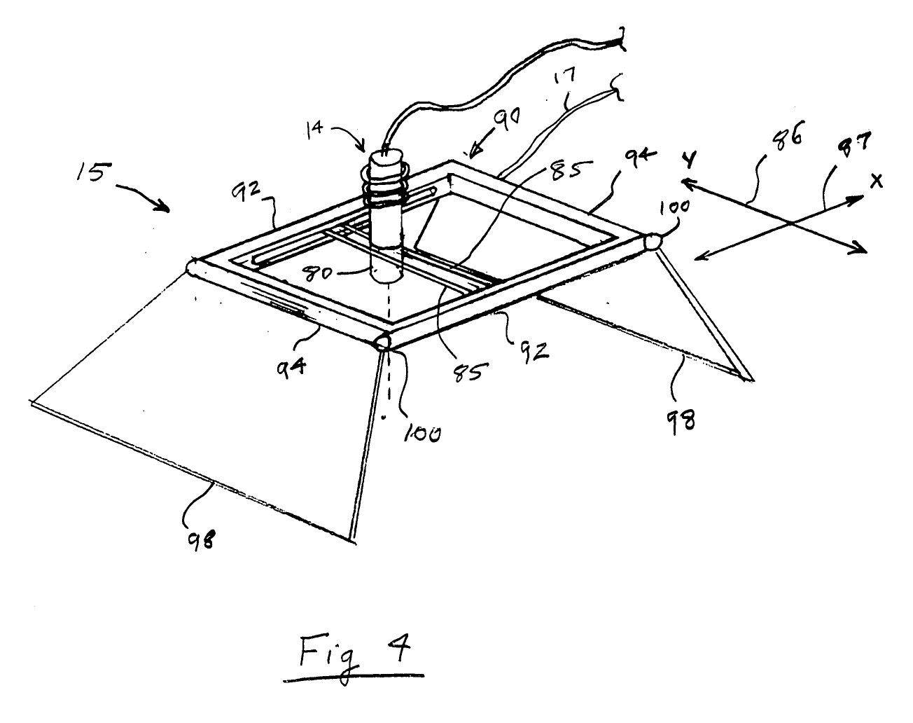

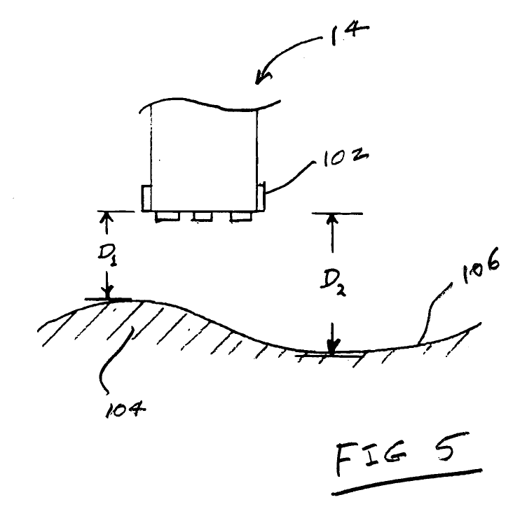

As shown in FIG. 3A, laser resonator 52 may include a housing 68. One or more diodes 70 (see FIG. 4) may be positioned within the housing 68 such that a beam emanating from each diode 70 may be directed outwardly from the housing 68, and such that the diode 70 is electrically connected for receiving electric current through an electrode 72 connected to wiring 51 that extends longitudinally through the hollow interior of tube 50 (FIG. 2). In the particular embodiment shown in FIG. 3B, five diodes 70 are shown by way of example only.

Each diode 70 may have peak emissive output at 3400 nm (3.4 μm) producing a low level reactive laser light having, and operate at a power output level of less than 100 mw. Other wavelengths may be utilized, depending on many variables, such as power ratings, degree of stimulation desired, and number of diodes. These diodes have wavelengths ranging from the near-infrared region of the electromagnetic spectrum, i.e. approximately 2500 nm, upward through the midregion of approximately 3000 nm to approximately 8000 nm, and beyond. Many types of diode semiconductor lasers may be used to produce the foregoing wavelengths, e.g., Nd:YAG, CO₂, Helium Neon, GaAs or the like. By way of example and not limitation, it has been found that a light-emitting diode such as model LED34-05, manufactured by the Boston Electronics Corporation, Brookline, Mass., may be used in this capacity to generate a beam having a wavelength of 3400 nm.

As shown in FIGS. 3A and 3B, a lens 74 may be positioned at one end of the housing 68 in the path of the generated laser light for focusing the light onto tissue treatment areas of, for example, 0.5 mm² to 2 mm², and to produce in the treatment areas an energy density in the range of from about 0.01 joules/mm² to about 0.15 joules/mm². The lens 74 may be adjusted to determine depth and area of absorption by those skilled in the art, without undue experimentation.

Referring now to FIG. 4, a hands-free device 15 is shown according to one embodiment of the invention. The hands-free device 15 may removeably receive a wand 14 in a cradle 80 supported by rails 85 for articulation over the surface of a patient's skin. The wand 14 may be maintained by the rails 85 in a vertical relationship with respect to a rectangular frame 90. The wand 14, cradle 80, and rails 85 may be supported by the rectangular frame 90, which is comprised of two articulation members 92 aligned in spaced, parallel relationship by static members 94 affixed to the ends of the articulation members 92. The frame 90 may be supported by legs 98 connected to the static members 94 by hinges 100. When the legs 98 are positioned as desired, the hinges 100 may be of a type that can be constrained from further movement by locking mechanisms well known to the art, such as screws or levers for frictionally constraining the hinge 100 in place. In this manner, the frame 90 may be placed over the patient's skin and vertically adjusted by means of the hinges 100 and legs 98, so that the end of the wand 14 can be statically positioned an arbitrary distance D1, D2 (FIG. 5) from the patient's skin.

The cradle 80 may contain an means therein (not shown) for moving the cradle 80 transversely in reciprocating movement along rails 85 in a Y-direction 86 as shown. The articulation members 92 may contain a means therein (not shown) for moving the cradle 80 and rails 85 as a unit in cooperative, reciprocating movement in an X-direction 87 that is orthogonal to the Y-direction 86 as shown. By moving the cradle 80 along the rails 85 in the Y-direction 86 while cooperatively moving the cradle 80 and rails 85 as a unit in the X-direction 87, it may be easily seen that any arbitrary path may be traversed by the wand 14 within the confines of the frame 90. The means for moving the cradle 80 and rails 85 may consist of standard devices that are well known to the art, such as, for example, small bidirectional electrical motors, under computer control, that are connected by pulleys, belts, or cables to the apparatus being moved. Other such means may consist of electromagnets having their magnetic fields under computer control for moving the apparatus. Other means may be used without departing from the scope of the invention.

Although the cradle 80 is shown as a cylindrical receptacle for receiving an end of the wand 14, other mechanisms and shapes may be used for this purpose without departing from the scope of the invention. Although parallel rails 85 are shown for supporting the cradle 80, the cradle 80 may be supported by a single bar or rail or other means that will allow the cradle 80 to be moved back and forth across the frame 90 without departing from the scope of the invention.

The hands-free device 15 may be connected to the biostimulation control unit 12 by a coaxial cable 17 (FIGS. 1 and 4). The coaxial cable 17 may provide both power to the means for moving the cradle 80 and rails 85 and control signals for directing the means to move the wand 14 in a designated path.

Referring now to FIG. 5, a capacitive proximity sensor may be provided to determine the distance between the end of the wand 14 and the surface of the patient's skin 104. A metallic ring 102 encircling an end of the wand 14, or a short coiled wire (like a spring), may be energized by small-signal oscillator operating at, for example, 1-2 Vrms at approximately 50 kHz, which may functionally serve as one plate of a capacitor. The skin surface 106 of the patient's grounded body may function as the other plate of the capacitor. This capacitor may form a portion of a capacitive bridge circuit, where a change in capacitance due to proximity of the skin sensor may be compared with a reference capacitor. The difference in AC voltage may be demodulated and filtered to a DC level that will be proportional to the different distances D1, D2 between the end of the wand 14 and the skin surface 106. Such impedance bridge circuits are commonly known in the art, and are often used in similar applications. Any of these circuit designs may be used without undue experimentation or departing from the scope of the invention. The demodulation and filtering circuit may be implemented using commercially-available, passive components, e.g. resistors and capacitors, and operational amplifiers packaged as integrated circuits.

The capacitive proximity sensor may be used when the biostimulation control unit 12 is in the automatic mode of operation. When operating in the automatic mode, the biostimulation control unit 12 may adjust the power sent to the wand 14 as it traverses over the surface of the patient's skin in response to the readings of the capacitive proximity sensor, so that a constant amount of radiation is delivered to the skin surface regardless of the distance the wand 14 is from the surface.

In operation, the switch 20 may be closed (i.e., turned on) to power up the biostimulation control unit 12, at which time the displays becomes illuminated, thereby indicating that the control unit is receiving power. The manual/automatic mode control 27 may be set depending upon whether it is desired to operate the biostimulation control unit 12 in the manual mode or the automatic mode.

If the manual mode of operation is selected by the manual/automatic mode control 27, then the desired manual parameters may be explicitly set up on the laser setting controls 26. The time control 28 may be set to specify a desired duration of time for laser treatment, which time is displayed on the countdown display 36a. The pulse/continuous mode control 32 may be set for specifying whether the laser light is to be generated in the continuous or the pulsed mode. If the pulsed mode is selected, then the duration of the pulse on-time/off-time may be specified and the pulses-per-second (and the pulse duty cycle if appropriate) may be displayed on the PPS display 40a. If the continuous mode is chosen instead, then the continuous mode display 40b is illuminated. It can be appreciated that the mode and the pulse time-on and time-off settings affect the intensity of the treatment provided. The amount of power may be further set by the power control 30 and displayed on the power display 38. It can be appreciated that the power, duration, and pulse intensity of treatment may thus be selectable by the biostimulation control unit 12 and may be determined by treatment protocols relating to the character of the tissue to be treated, the depth of penetration desired, the acuteness of the injury, and the condition of the patient. The audio volume control 46 may be adjusted to control the volume of the tone generated from the speaker 48. The tissue impedance display 42 may indicate an impedance value for tissue in contact with it and can be calibrated to a baseline set for the patient by applying the wand 14 to surrounding non-damaged tissue, and then, when the wand 14 is applied to the damaged tissue, an impedance value (much higher than the baseline) may be indicated and hopefully reduced over time, through treatment, to the baseline value.

After selections are made for the time, power, and mode (continuous wattage or pulsed at a selected intensity), the wand 14 may be directed into the calibration port 49 to verify the accuracy of the system. The wand 14 may then be manually applied to patient tissue for therapy. The foot pedal 22 and/or the switch 64 may be depressed to cause therapeutic laser light energy to be generated from the wand 14. As an indication that laser light energy is being generated, an audible tone may be generated from the speaker 32. In accordance with the foregoing specification of the laser diode 70, the laser light energy may be generated at a fundamental wavelength of about 3400 nm at an output power level of from about 100 mw to about 800 mw. In other implementations the laser light wavelength may range between about 2500 nm to about 10000 nm, at a total combined power of up to 1000 mw for all diodes 70 in the wand 14.

If the automatic mode of operation is selected by the manual/automatic mode control 27, then a set of parameters comprising a protocol may be selected from the protocols presently stored in the biostimulation control unit 12 using the programmed settings control 44. The hands-free device 15 must be connected to the biostimulation control unit 12 via the coaxial cable 17 and the wand 14 must be inserted into the cradle 80 in order to operate in the automatic mode. In addition to the parameters contained in the protocol, directions for moving the cradle 80 within the frame 90 must also be accessed. These directions may be developed on other support systems and read into the biostimulation control unit 12 through the input/output device 11. The directions may describe a path to be traversed by the cradle 80 containing the wand 14 and contain such additional parameters as a set of points defining a path to be taken by the cradle 80, in terms of x-y coordinates; a time value to be used in moving between consecutive points; a loiter time for each point; and a repetition number for the number of times that the directions will be repeated.

The generated laser optical energy may be applied to regions of the body where desired therapeutic results are desired, such as decreased muscle spasms, increased circulation, decreased pain, or enhanced tissue healing. The surface of the tissue in the region to be treated may be demarcated to define an array of grid treatment points, each of which points identifies the location of an aforementioned small treatment area. Each small treatment area may be irradiated with the laser beam light to produce the desired therapeutic effect. Because laser light is coherent, a variable energy density of the light of from about 0.01 to 0.15 joules/mm² may be obtained as the light from the one or more diodes 70 passes through the lens 74 and converges onto each of the small treatment areas. The energy of the optical radiation may be controlled by the power control 30 and applied (for durations such as 1 minute to 3 minutes, continuous wattage or pulsed, for example) as determined by treatment protocols, to cause the amount of optical energy absorbed and converted to heat to be within a range bounded by a minimum absorption rate sufficient to elevate the average temperature of the irradiated tissue to a level which is above the basal body temperature, but which is less than the absorption rate at which tissue is converted into a collagenous substance. The laser beam wavelength, spot or beam size, power dissipation level, and time exposure may thus be carefully controlled to produce in the irradiated tissue a noticeable warming effect which may also be limited to avoid damaging the tissue from thermal effects.

The present invention has several advantages. For example, by using an In:GaAs diode laser to generate the laser beam energy, the laser source can be made sufficiently small to fit within the wand 14, thereby obviating the need for a larger, more expensive laser source and the fiber optic cable necessary to carry the laser energy to the treatment tissue. The In:GaAs diode laser can also produce greater laser energy at a higher power dissipation level than lasers of comparable size. Furthermore, construction of the wand 14 including the fins 58 may provide for the dissipation from the wand 14 of the heat generated by the laser source.

A further advantage is that therapeutic treatment by the foregoing low level reactive laser system has been shown to reduce pain in soft tissue, reduce inflammation, and enhance healing of damaged tissue by the stimulation of microcirculation, without subjecting the living tissue to damaging thermal effects. This phenomenon is due to certain physiological mechanisms in the tissue and at the cellular level that occur when the above process is used. In the evaluation of the microcirculatory system, for example, it has been demonstrated that the blood vessel walls possess photosensitivity. When the blood vessel walls are exposed to laser irradiation as set forth above, the tonus is inhibited in smooth myocytes, thus increasing the blood flow in the capillaries. Other effects which have been observed are: peripheral capillary neovascularization, reduction of blood platelet aggregation, reduction of O₂ from the triplet to the singlet form which allows for greater oxygenation of the tissue, reduction of buffer substance concentration in the blood, stabilization of the indices of erythrocyte deformation, reduction of products of perioxidized lipid oxygenation of the blood. Other effects which have been observed are increased index of antithrombin activity, stimulation of the enzymes of the antioxidant system such as superoxide dismutase and catalase. An increase in the venous and lymph and outflow from the irradiated region has been observed. The tissue permeability in the area is substantially enhanced. This assists in the immediate reduction of edema and hematoma concentrations in the tissue. At the cellular level, the mitochondria have also been noted to produce increased amounts of ADP with subsequent increase in ATP. There also appears to be an increased stimulation of the calcium and sodium pumps at the tissue membrane at the cellular level.

At the neuronal level, the following effects have been observed as a result of the foregoing therapeutic treatment. First, there is an increased action potential of crushed and intact nerves. The blood supply and the number of axons is increased in the irradiated area. Inhibition of scar tissue is noticed when tissue is lazed. There is an immediate increase in the membrane permeability of the nerve. Long term changes in the permeability of calcium and potassium ions through the nerve for at least 120 days have been observed. The RNA and subsequent DNA production is enhanced. Singlet O₂ is produced which is an important factor in cell regeneration. Pathological degeneration with nerve injury is changed to regeneration. Both astrocytes and oligodedrocytes are stimulated which causes an increased production of peripheral nerve axons and myelin.

Phagocytosis of the blood cells is increased, thereby substantially reducing infection. There also appears to be a significant anti-inflammatory phenomena which provides a decrease in the inflammation of tendons, nerves, bursae in the joints, while at the same time yielding a strengthening of collagen. There is also an effect on the significant increase of granulation tissue in the closure of open wounds under limited circulation conditions.

Analgesia of the tissue has been observed in connection with a complex series of actions at the tissue level. At the local level, there is a reduction of inflammation, causing a reabsorption of exudates. Enkephalins and endorphins are recruited to modulate the pain production both at the spinal cord level and in the brain. The serotnogenic pathway is also recruited. While it is not completely understood, it is believed that the irradiation of the tissue causes the return of an energy balance at the cellular level which is the reason for the reduction of pain.

As can be seen, the invention provides a means for directing a diode laser wand across a portion of tissue in a repeatable manner, to enable consistent tracking of results. It should be understood, of course, that the foregoing relates to exemplary embodiments of the invention and that modifications may be made without departing from the spirit and scope of the invention as set forth in the following claims.

We claim:

1. A diode laser irradiation system for therapeutically treating biological tissue of a patient without exposing the tissue to damaging thermal effects, the system comprising:
a wand comprising one or more diode lasers for irradiating the tissue with coherent optical energy focused to an area in the range of about 0.5 mm² to about 2 mm² at a power output level of less than 1000 milliwatts;
a means for moving the wand over the tissue in a repeatable manner; and
one or more laser setting controls for operating the diode lasers to achieve a rate of absorption and conversion to heat in the irradiated tissue in a range between a minimum rate sufficient to elevate the average temperature of the irradiated tissue to a level above the basal body temperature of the subject, and a maximum rate which is less than the rate at which the irradiated tissue is converted into a collagenous substance.

2. The system described in claim 1, wherein the laser setting control is selected from a group that includes
a time control for setting the irradiation treatment time;
a power control for setting the power level of the diode laser;
a pulse/continuous mode control for setting the diode laser to operate in a continuous wattage mode of operation or in a pulsed wattage mode of operation;
a programmed setting control for saving and recalling selected laser settings; and
an impedance control for calibrating an impedance reading of the tissue;

3. The system described in claim 1, further comprising a pulse/continuous mode control for setting the diode laser to operate in a continuous wattage mode of operation or in a pulsed wattage mode of operation, wherein the pulse/continuous mode control selects the number of light pulses-per-second emitted by the laser diode when operating in the pulsed wattage mode.

4. The system described in claim 1, the system further comprising an impedance control for calibrating an impedance reading of the tissue, and the the wand further comprising an impedance sensor for contact with the tissue for measuring impedance of the tissue being treated.

5. The system described in claim 1, further comprising a time display for displaying the treatment time remaining for a treatment time selected using the setting controls.

6. The system described in claim 1, further comprising a power display for displaying a treatment power output selected using the setting controls.

7. The system described in claim 1, wherein the diode laser is an Indium-doped Gallium Arsenide diode laser.

8. The system described in claim 1, further comprising a calibration port for calibrating the settings of the diode laser by placing the wand in proximity to the port.

9. A diode laser irradiation system for therapeutically treating biological tissue of a patient without exposing the tissue to damaging thermal effects, the system comprising:
a wand comprising one or more diode lasers for irradiating the tissue with coherent optical energy focused to an area in the range of about 0.5 mm² to about 2 mm² at a power output level of less than 1000 milliwatts, the coherent optical energy emitted by each diode laser having a wavelength in a range of from about 2500 nm to about 10,000 nm; and
one or more laser setting controls for operating the diode lasers to achieve a rate of absorption and conversion to heat in the irradiated tissue in a range between a minimum rate sufficient to elevate the average temperature of the irradiated tissue to a level above the basal body temperature of the subject, and a maximum rate which is less than the rate at which the irradiated tissue is converted into a collagenous substance.

10. The system described in claim 9, wherein the coherent optical energy emitted by the diode laser has a wavelength of about 3400 nm.

11. The system described in claim 9, wherein the laser setting control is selected from a group composed of
a time control for setting the irradiation treatment time;
a power control for setting the power level of the diode laser;
a pulse/continuous mode control for selectively setting the diode laser to operate in a continuous wattage mode and in a pulsed wattage mode;
a programmed setting control for saving and recalling selected laser settings; and
an impedance control for calibrating an impedance reading of the tissue.

12. The system described in claim 9, the system further comprising a means for moving the wand over the tissue in a repeatable manner.

13. The system described in claim 12, wherein the wand further comprises a proximity sensor for determining a distance between an end of the want and the tissue of the patient.

14. The system described in claim 9, wherein the diode laser is an Indium-doped Gallium Arsenide diode laser.

15. A method for treating biological tissue of a subject using a diode laser irradiation system, the method comprising:
positioning a hands-free device over the tissue, the hands-free device comprising a wand containing one or more diode lasers for irradiating the tissue with coherent optical energy focused to an area in the range of about 0.5 mm² to about 2 mm² at a power output level of less than 1000 milliwatts and a means for moving the wand in a repeatable pattern over the tissue;
operating the diode laser to achieve a rate of absorption and conversion to heat in the irradiated tissue in a range between a minimum rate sufficient to elevate the average temperature of the irradiated tissue to a level above the basal body temperature of the subject, and a maximum rate which is less than the rate at which the irradiated tissue is converted into a collagenous substance; and
moving the wand over the tissue in a regular, repeatable pattern.

16. The method described in claim 15, further comprising the step of
accessing a stored protocol containing parameters describing a path to be traversed by the wand and selectively setting the irradiation treatment time, the power level, and the pulse/continuous operating mode of the diode laser according to the protocol.

17. The method described in claim 15, wherein the coherent optical energy emitted by the diode laser has a wavelength in a range of from about 2500 nm to about 10,000 nm.

18. The method of claim 15, wherein the coherent optical energy emitted by the diode laser has a wavelength of about 3400 nm.

19. The method described in claim 15, further comprising the step of
adjusting the power sent to the wand as it traverses over a surface of the tissue of the patient in response to readings of a capacitive proximity sensor in the wand, wherein a constant amount of radiation is delivered to the surface regardless of the distance the wand is from the surface.
